(19) United States
(12) Patent Application Publication
(10) Pub. No.: US 2018/0250163 A1
(43) Pub. Date: Sep. 6, 2018

(54) APPARATUS FOR OCULAR TREATMENT AND METHOD FOR OPERATING APPARATUS FOR OCULAR TREATMENT

(71) Applicant: LUTRONIC VISION INC., Burlington, MA (US)

(72) Inventor: Hee Chul LEE, Goyang (KR)

(21) Appl. No.: 15/756,553

(22) PCT Filed: Aug. 31, 2016
(86) PCT No.: PCT/KR2016/009718

§ 371 Date: Feb. 28, 2018

(30) Foreign Application Priority Data
Aug. 31, 2015 (KR) 10-2015-0122479

(51) Int. Cl. A61F 9/008; A61F 9/009

(52) U.S. Cl. CPC A61F 9/0084; A61F 9/009; A61F 2009/00851; A61F 2009/0087; A61F 9/00814

(57) ABSTRACT

An apparatus for ocular treatment according to the present invention comprises: a beam generation unit for generating a therapeutic beam; a beam transfer unit comprising a first lens part capable of forming an optical path through which the therapeutic beam oscillated from the beam generation unit travels in the ocular fundus, and of varying the focal length so that the beam is transferred in a predetermined spot size in the ocular fundus; and a control unit, connected to the beam transfer unit, for controlling the focal length of the therapeutic beam of the first lens part.

### TECHNICAL FIELD

The present invention relates to an ophthalmic treatment apparatus and a method of driving the ophthalmic treatment apparatus, and more particularly, to an ophthalmic treatment apparatus for treating a lesion of the eyeball by irradiating the lesion of the eyeball with a therapeutic beam such as a laser, and a method of driving the ophthalmic treatment apparatus.

### BACKGROUND ART

An ophthalmic therapeutic apparatus for treating the eyeball are used to treat lesions of the cornea, the crystalline lens, the retina and the like. Specifically, the ophthalmic treatment apparatus can be used for adjustment of the refractive index of the cornea, the crystalline lens due to cataracts, glaucoma treatment, and macular degeneration of the retina.

Such an ophthalmic treatment apparatus irradiates a treatment region of the eyeball, that is, a lesion region with a therapeutic beam. Here, the therapeutic beam irradiated from the ophthalmic treatment apparatus may be a laser having a wavelength band suitable for lesion treatment.

On the other hand, in the conventional art, for example, existing equipment has at least 4 to 6 or more lenses arranged in the equipment to constitute a beam transfer unit. Specifically, in order to produce various spot sizes necessary for treatment, the convention art has a structure of a plurality of independent lenses arranged corresponding to respective spot sizes for generating a beam.

As such, in the case the spot sizes of the therapeutic beam are individually generated using a relatively large number of lenses, there occur undesired lens tolerances caused by the complicated structure. Accordingly, it is difficult to cope with the tolerances at the time of treating the patient, and there is a problem that the mass-produce of the laser unit is impossible.

### DISCLOSURE

### Technical Problem

In the apparatus for treating lesions of the eyeball with a laser, an object of the present invention is to provide an ophthalmic treatment apparatus that improves a lens system of the treatment apparatus so that the tolerance and the focal length of the lens can be easily varied, and a method of driving the ophthalmic treatment apparatus.

### Technical Solution

An ophthalmic treatment apparatus according to the present invention may include a beam generation unit for generating a therapeutic beam; a beam transfer unit including a first lens part capable of forming an optical path through which the therapeutic beam oscillated from the beam generation unit proceeds to the eyeball, and of varying a focal length of the therapeutic beam so that the therapeutic beam is transferred to the eyeball in a predetermined spot size; and a control unit, connected to the beam transfer unit, for controlling the focal length of the therapeutic beam in accordance with the first lens part.

In addition, the control unit may generate a variable signal and transmits the variable signal to the first lens part so that the focal length of the therapeutic beam is varied at the first lens part. In addition, the first lens part may vary the focal length by receiving an amount of current or voltage corresponding to the variable signal transmitted from the control unit.

In addition, the first lens part may include an electrical lens.

Also, the therapeutic beam of a spot size varied through the first lens part may be transferred to a cornea region or a retina region of the eyeball.

In addition, a diameter of the spot size in which the therapeutic beam may be transferred to the retinal region is 40 to 1200 μm, and a diameter of the spot size in which the therapeutic beam may be transferred to the corneal region is 300 to 450 μm.

In addition, the ophthalmic treatment apparatus may further include a contact lens for guiding the therapeutic beam transferred from the beam transfer unit to the eyeball.

Also, the beam transfer unit may further include a second lens part for converging the therapeutic beam with the focal length varied by the first lens part.

Further, the ophthalmic treatment apparatus may further include an image unit for photographing a treatment region in the eyeball to form an image thereof in order to set an irradiation position of the therapeutic beam to be transferred to the treatment region of the eyeball.

A method of driving an ophthalmic treatment apparatus according to the present invention may include the steps of: generating a therapeutic beam in the beam generation unit; varying a focal length in the first lens part such that the therapeutic beam forms a predetermined spot size in the eyeball; and irradiating the therapeutic beam of the controlled spot size into the eyeball.

In addition, the step of irradiating the controlled therapeutic beam into the eyeball may include using a second lens part that converges the therapeutic beam of which focal length has been varied by the first lens part to transfer the therapeutic beam thereto.

Also, the step of controlling the spot size of the therapeutic beam to be formed may include generating a variable signal based on an amount of current or voltage in the control unit; and transmitting the variable signal to the first lens unit so that the spot size of the therapeutic beam is varied.

Further, the method of driving an ophthalmic treatment apparatus may further include setting an irradiation position of the therapeutic beam transferred to a treatment region of the eyeball prior to the step of controlling the predetermined spot size to be formed in the eyeball.

The details of other embodiments are included in the detailed description and the drawings.

### Advantageous Effects

The ophthalmic treatment apparatus and the method of driving the ophthalmic treatment apparatus according to the present invention have an advantageous effect that can improve the treatment efficiency of the eyeball by providing a lens that can variously vary the focal length according to the spot size of the beam transferred into the eyeball for treatment.

In addition, the ophthalmic treatment apparatus according to the present invention can maximize structural simplification and mass productivity by using a single lens that can replace a plurality of lenses.

### DESCRIPTION OF DRAWINGS

FIG. 1 is a control block diagram of an ophthalmic treatment apparatus according to an embodiment of the present invention.

FIG. 2 is a schematic configuration diagram of an ophthalmic treatment apparatus according to an embodiment of the present invention.

FIG. 3 is a view showing a spot size in the cornea or the retina in accordance with variation of a focal length of a first lens part in an ophthalmic treatment apparatus according to an embodiment of the present invention.

FIG. 4 is a flowchart showing a method of driving the ophthalmic treatment apparatus according to an embodiment of the present invention.

### MODE FOR INVENTION

Hereinafter, an ophthalmic treatment apparatus and a method of driving the ophthalmic treatment apparatus according to an embodiment of the present invention will be described in detail with reference to the accompanying drawings. However, it is to be understood that the terms or words disclosed below in the present embodiment should not be construed as being ordinary or dictionary definitions, but should be construed as the meaning and concept consistent with the technical idea of the present invention based on the principle that the inventor may appropriately define the concept of a term to describe his own invention in the best way.

Therefore, the embodiments described in the present specification and the configurations shown in the drawings are merely the most preferred embodiment of the present invention and are not intended to represent all of the technical ideas of the present invention. Therefore, it should be understood that various equivalents and modifications may be made to be substituted with the present embodiments and configurations at the time of filing the present application.

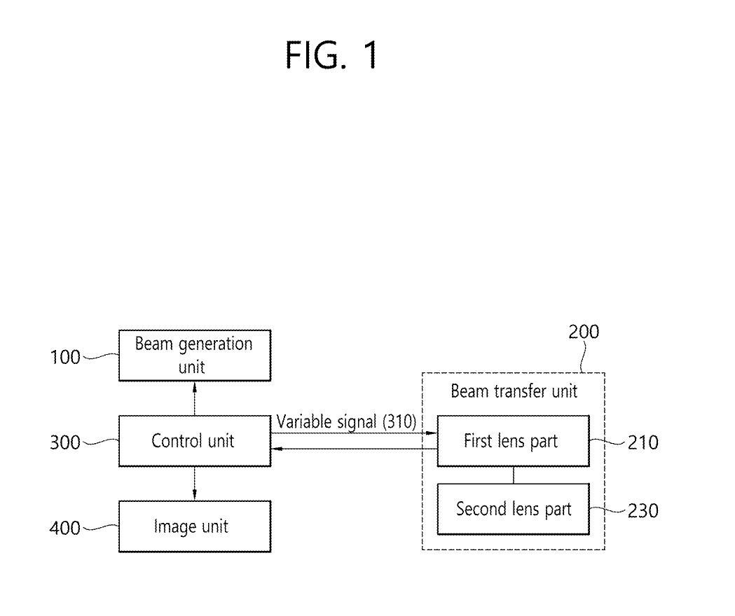

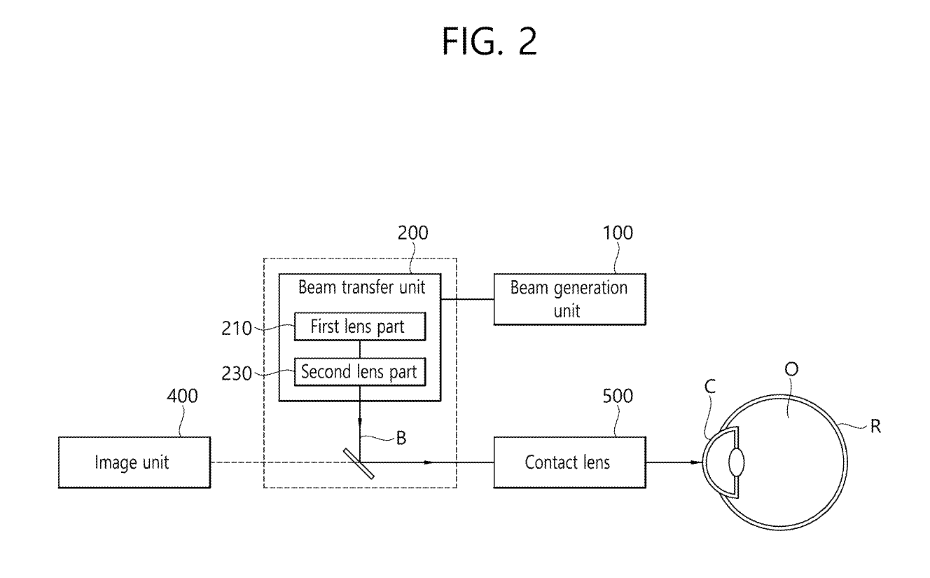

FIG. 1 is a control block diagram of an ophthalmic treatment apparatus according to an embodiment of the present invention. FIG. 2 is a schematic configuration diagram of an ophthalmic treatment apparatus according to an embodiment of the present invention.

As shown in FIGS. 1 and 2, an ophthalmic treatment apparatus 10 according to the embodiment of the present invention may include a beam generation unit 100, beam transfer unit 200, an image unit 400, a control unit 300, and a contact lens 500. Although the ophthalmic treatment apparatus 10 according to the present invention is described below as an ophthalmic treatment apparatus 10 for treating the retina R, it is, of course, also may be used to treat various ophthalmic regions such as the cornea C and the crystalline lens other than the retina R.

The beam generation unit 100 generates a therapeutic beam B capable of treating a lesion of the retina R or the cornea C. The therapeutic beam B generated from the beam generation unit 100 may be a laser having a certain wavelength band. The beam generation unit 100 may be constituted by a laser diode or the like so that a laser having a certain wavelength band can be generated as the therapeutic beam B. The wavelength band of the therapeutic beam B generated from the beam generation unit 100 may be 532 nm to 1064 nm. However, the wavelength band of the therapeutic beam B generated from the beam generation unit 100 may be less than 532 nm or more than 1064 nm, depending on the tissue characteristics of the lesion region to be treated.

The image unit 400 may be provided to photograph an image of the eyeball O. The image unit 400 can irradiate the eyeball with light and can take an image of the surface of the eyeball O or conduct a tomographic scan of the eyeball by using the reflected light. Here, an optical path through which light proceeds may be formed by using constituent elements such as optical lenses provided in the beam transfer unit 200, or by disposing a discrete optical element separate from the beam transfer unit 200. In the case of conducting the tomographic scan of the eyeball O with the image unit 400, Optical Coherence Tomography (OCT) or the like may be used as an example. In the case of using OCT, the tomographic scan of the eyeball O may be conducted along an axis of light to which the therapeutic beam B is irradiated.

Although not shown in the present invention, an image analysis unit (not shown) connected to the image unit 400 may be disposed inside the ophthalmic treatment apparatus 10. The image analysis unit may analyze the image of the eyeball O photographed from the image unit 400, identify regions requiring treatment before treatment is performed, and then confirm whether or not the treatment is appropriately provided after treatment.

The beam transfer unit 200 receives the therapeutic beam B generated from the beam generation unit 100 and transfers the therapeutic beam B to the retina R or the cornea C which is a treatment region.

The beam transfer unit 200 may include a first lens part 210 for generating a predetermined spot size in the retina R or the cornea C which requires treatment. Also, the beam transfer unit 200 may include a second lens part 230 for converging the therapeutic beam of which spot size has been adjusted by the first lens part 210.

The first lens part 210 is disposed inside the beam transfer unit 200 and can form an optical path through which the therapeutic beam B oscillated from the beam generation unit 100 proceeds to the eyeball O. At this time, the first lens part 210 can vary a focal length so that the therapeutic beam B transferred to the retina R of the fundus or the cornea C forms a predetermined spot size.

In the conventional art, for example, existing equipment has at least 4 to 6 or more lenses arranged in the equipment to constitute a beam transfer unit. Specifically, in order to produce various spot sizes necessary for treatment, the convention art has a structure of a plurality of independent lenses arranged corresponding to respective spot sizes for generating a beam. As such, in the case of the equipment using a relatively large number of lenses, there are disadvantages that tolerances between lenses occur and that it is difficult to cope with the tolerances at the time of treating the patient. Also, there is a problem that the mass-produce of the laser unit is impossible due to the complicated structure.

However, since the beam transmission unit 200 of the present invention can adjust the focal length and can generate various spot sizes of the therapeutic beam B transferred to the eyeball in real time with only one of the single first lens part 210, so that the patient's lesion can be treated with a simple structure as compared to the convention art. Specifically, the first lens part 210 is connected to the control unit 300, and can change the focal length in various ways according to a variable signal 310 controlled by the control unit 300. The spot size of the therapeutic beam transferred to the cornea C or the retina R according to the change of the focal length can be variously controlled. The control unit 300 will be described below.

As one embodiment of the first lens 210. an electrical lens may be used. The electron lens receives a current or a voltage and influences the trajectory of electrons inside the electron lens, so that a path of advancing light can be varied. The electron lens can be divided into an electrostatic lens using an electric field and a magnetic lens using a magnetic field.

The second lens part 230 is disposed inside the beam transfer unit 200 and the therapeutic beam B of which focal length has been varied by the first lens part 210 can be converged. The second lens part 230 may be provided as a convex lens for conversing the therapeutic beam B.

Then, the therapeutic beam B converged by the convex lens passes through the inside of the beam transfer unit 30 and proceeds to the contact lens 500.

The control unit 300 is connected to the beam transfer unit 200, and performs a function of controlling the focal length of the therapeutic beam B in accordance with the first lens part 210.

The control unit 300 can transmit a variable signal 310, which can vary the focal length, to the first lens part 210. In the case an electrical lens is used as the first lens part 210 according to an embodiment of the present invention, the variable signal 310 may a signal for controlling an amount of current or voltage supplied to the first lens part 210. For example, the control unit 300 may generate a variable signal 310 corresponding to a predetermined amount of current or voltage to generate a predetermined focal length and a predetermined spot size in accordance with the predetermined focal length.

When the variable signal 310 is transmitted to the first lens part 210, the first lens part 210 receives the predetermined amount of current or voltage and can vary the focal length to a region requiring treatment. Thereafter, according to the variation of the focal length, the therapeutic beam B corresponding to an appropriate spot size can be transferred to a region of the retina R and the cornea C requiring treatment.

Also, in addition to the predetermined method, in order to transfer the laser with various spot sizes to the region in real time according to the patient or the lesion during treatment, the control unit 300 may transmit a corresponding variable signal 310 to the first lens while changing it in real time.

Also., the control unit 300 can control operation of the image unit 400 that photographs an image of the eyeball O.

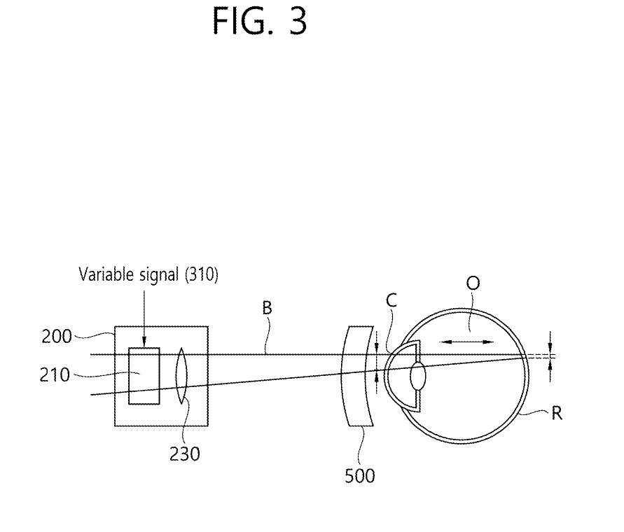

FIG. 3 is a view showing a diameter of the spot size of the therapeutic beam transferred to the cornea or the retina in accordance with the variation of the focal length of the first lens part according to an embodiment of the present invention.

Table 1 shows simulated results in the case the therapeutic beam B is transferred to the cornea C and the retina R.

TABLE 1

| Spot size at | Spot size at | Needed Focal length of |
|---|---|---|
| Retina (μm) | Cornea (μm) | Electrical lens (mm) |
| 49 | 413 | 40.07 |
| 102 | 408 | 41.08 |
| 206 | 398 | 41.76 |
| 296 | 389 | 42.37 |
| 506 | 369 | 43.85 |
| 1004 | 321 | 47.86 |

Referring to FIG. 3 and Table 1, according to an embodiment of the present invention, the amount of change in the spot size of the therapeutic beam B transferred to the retina R or the cornea C is shown in accordance with variation of the focal length at the first lens part 210.

The present embodiment is given as an example in the case while the therapeutic beam B is to have spot sizes of 50 μm, 100 μm, 200 μm, 300 μm, 500 μm, and 1000 μm in diameter at the retina R, spot sizes in diameter at the cornea is to become equal to or greater than 300 μm (which may be to have a larger size depending on the design).

In Table 1, it is shown that when the diameter of the spot size transferred to the retina R is increased to 50 μm to 200 μm, the amount of change in the spot size in the cornea C is insignificantly reduced. Then, when the diameter of the spot size transferred to the retina R is equal to or greater than 300 μm, the amount of change in the spot size in the cornea C begins to increase slightly, and when the spot size transferred to the retina R is 1000 μm, the amount of change in the spot size in the cornea C shows a rate of change increased by 20% compared with the previous numerical value.

In the treatment of the retina R, the therapeutic beam B must pass through the cornea C and be delivered to the retina R region in a necessary spot size. Therefore, it is preferable that the treatment apparatus 10 transfer an amount of heat energy necessary for treatment to the retina R and transfers a minimum energy to the cornea C. According to the above described embodiment, the amount of thermal energy transferred to each region can be confirmed by the amount of change depending on the spot size transferred to the cornea C according to the amount of change in the spot size transferred to the retina R. Specifically, referring to the amount of change in the spot size transferred to the cornea C with respect to the amount of change in the spot size capable of treating the lesion of the retina R, it has been confirmed from an experimental result that the amount of change in the spot size transferred to the cornea C, that is, the transferred energy and the width of change show spot sizes in accordance with the generally acceptable energy and the width of change in terms of biostability.

Also, it has been confirmed from the experimental result that the amount of change in the focal length of the first lens part 210 is a numerical value that is sufficiently controllable by the first lens part 210 and the control unit 300.

The diameter of the spot size in which the therapeutic beam B of the present invention is transferred to the retinal R region corresponds to 40-1200 μm and has a wide range of bands capable of treating a plurality of lesions in the retinal R. The diameter of the spot size transferred to the cornea C region corresponds to 300-450 μm, so that while treating the retina R, energy with safe bands can be delivered to the cornea C without damage to the cornea C.

In conclusion, the amount of change in the spot size in the retina R according to the focal length by the first lens part 210 could appropriately perform the treatment of the patient's lesion. Also, since energy that is more than necessary is not transferred to the cornea C, safe treatment could be performed. Further, it has been found that the present invention is capable of variously controlling the spot size according to the focal length variation by only one of the first lens part 210, thereby having advantageous effects of performing convenient and quick treatment by a simple operation and a simple configuration.

Of course, in addition to the above embodiment, the spot size transferred to the cornea C and the retina R may have a larger value depending on the design of the apparatus and the adjustment of the first lens part 210.

Based on the variable signal 310 controlled by the control unit 300, the therapeutic beam B that has passed through the beam transfer unit 200 can be guided to the contact lens 500 for transfer to intraocular legions.

The contact lens 500 is disposed between the beam transfer unit 200 and the eyeball O and contacts with the eyeball O to ensure visibility of the retina R. That is, the contact lens 500 is in contact with the cornea C of the eyeball O for the operator to see the retina R of the fundus. The contact lens 500 may be basically provided in a conical shape.

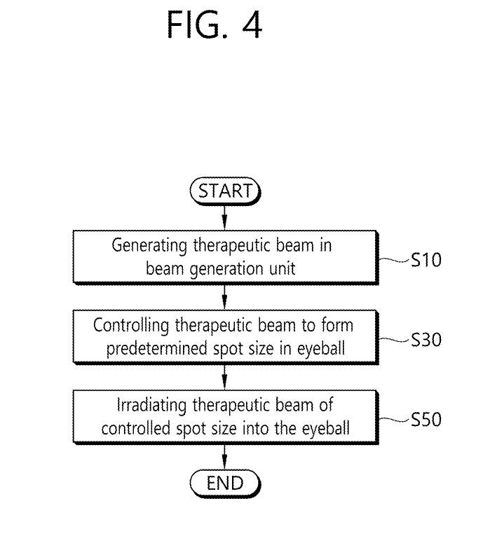

A driving method for irradiating the therapeutic beam B of the ophthalmic treatment apparatus 10 of the present invention with such a structure will be described with reference to FIG. 4.

FIG. 4 is a flowchart showing a method of driving the ophthalmic treatment apparatus according to an embodiment of the present invention.

First, an image can be formed by photographing a treatment region in the eyeball O by using an image unit 400. Then, the irradiation position of a therapeutic beam B can be set for the treatment region of the eyeball O.

Then, a step of generating a therapeutic beam B in a beam generation unit 100 may be performed so that the therapeutic beam B is transferred to the predetermined irradiation position (S10). The step of generating the therapeutic beam B in the beam generation unit 100 may be performed after adjusting a focal length of a lens in a beam transfer unit 200 to be described later.

The therapeutic beam B generated from the beam generation unit 100 may be a laser having a certain wavelength band. The beam generation unit 100 may be constituted by a laser diode or the like so that a laser having a certain wavelength band can be generated as the therapeutic beam B.

The method of setting the irradiation position of the therapeutic beam B may be performed by a step of controlling a spot size according to variation of the focal length (S30). First, a control unit 300 can generate a variable signal 310 applied to vary the focal length, and transfer it to a first lens part 210 of the beam transfer unit 200. The variable signal 310 may refer to a digital signal or the like generated by the control unit 300 so that a user can set a desired focal length by controlling an amount of current or voltage supplied to the first lens part 210.

Then, the first lens part 210 of the beam transfer unit 200 may generate an appropriate spot size necessary for the treatment region according to the variable signal 310 received from the control unit 300. According to the driving method of the beam transfer unit 200 and the control unit 300, the adjustment of the spot size through the variation of the focal length may be performed in a predetermined manner, or, of course, the spot size through the variation of the focal length may be adjusted while confirming the patient's treatment lesion in real time.

The diameter of the spot size in which the therapeutic beam B is transferred to the retina R region is 40-1200 μm and the diameter of the spot size in which the therapeutic beam B is transferred to the cornea C region is 300-450 μm.

In the conventional art, at least 4-6 lenses are arranged in a row to constitute a beam transfer unit, and a relatively large number of lenses are used to generate the spot size of the therapeutic beam B. For this reason, there occurs undesired lens tolerances caused by the complicated structure, and it is difficult to cope with the tolerances, and there is a problem that effective treatment cannot be performed during the treatment of the patient.

However, according to the mutually associated configuration and operation method of the first lens part 210 and the control unit 300 of the present invention, the focal length adjustment can be performed with only one of the first lens parts 210. In addition, since the spot size of the therapeutic beam B transferred in the fundus can be variously generated according to the varied focal length by the first lens part 210, the lesion of the patient can be treated with a simple structure as compared to the convention art. As a result, the first lens part 210 is connected to the control unit 300, there is an advantageous effect in that the focal length can be changed variously according to the variable signal 310 controlled by the control unit 300.

Then, the therapeutic beam B generated in the beam generation unit 100 may be irradiated, along the optical path formed by the beam transfer unit 200, onto the treatment region of the fundus. A contact lens 500 is disposed in contact with the cornea C of the eyeball O, so it can guide the therapeutic beam B transferred from the beam transfer unit 200 to the eyeball O to help the treatment proceed.

Therefore, as described above, while the therapeutic beam B is being irradiated onto the treatment region of the eyeball O, the spot size according to the irradiation position of the therapeutic beam B, that is, the focal length can be adjusted in real time, and by control in real time, an appropriate amount of energy can be easily provided to the lesion of the patient.

As above, while the embodiments of the present invention have been described with reference to the accompanying drawings, it will be understood by a person with ordinary skill in the art that the present invention may be embodied in many other specific forms without departing from the technical spirit or essential features of the present invention. Therefore, it is to be understood that the above-described embodiments are illustrative in all aspects and not restrictive. The scope of the present invention is defined by the claims to be described rather than the detailed description, and all changes or modifications derived from the meaning and scope of the claims and their equivalents are to be construed as being included within the scope of the present invention.

1. An ophthalmic treatment apparatus comprising:
a beam generation unit for generating a therapeutic beam;
a beam transfer unit comprising a first lens part capable of forming an optical path through which the therapeutic beam oscillated from the beam generation unit proceeds to the eyeball, and of varying a focal length of the therapeutic beam so that the therapeutic beam is transferred to the eyeball in a predetermined spot size; and
a control unit, connected to the beam transfer unit, for controlling the focal length of the therapeutic beam in accordance with the first lens part.

2. The apparatus according to claim 1, wherein the control unit generates a variable signal and transmits the variable signal to the first lens part so that the focal length of the therapeutic beam is varied at the first lens part.

3. The apparatus according to claim 2, wherein the first lens part varies the focal length by receiving an amount of current or voltage corresponding to the variable signal transmitted from the control unit.

4. The apparatus according to claim 2, wherein the first lens part comprises an electrical lens.

5. The apparatus according to claim 1, wherein the therapeutic beam of a spot size varied through the first lens part is transferred to a cornea region or a retina region of the eyeball.

6. The apparatus according to claim 5, wherein a diameter of the spot size in which the therapeutic beam is transferred to the retinal region is 40 to 1200 μm, and a diameter of the spot size in which the therapeutic beam is transferred to the corneal region is 300 to 450 μm.

7. The according to claim 1, further comprising a contact lens for guiding the therapeutic beam transferred from the beam transfer unit to the eyeball.

8. The apparatus according to claim 1, wherein the beam transfer unit further comprises a second lens part for converging the therapeutic beam with the focal length varied by the first lens part.

9. The apparatus according to claim 1, further comprising an image unit for photographing a treatment region in the eyeball to form an image thereof in order to set an irradiation position of the therapeutic beam to be transferred to the treatment region of the eyeball.

10. A method of driving an ophthalmic treatment apparatus, the method comprising the steps of:
generating a therapeutic beam in the beam generation unit;
varying a focal length in the first lens part such that the therapeutic beam forms a predetermined spot size in the eyeball; and
irradiating the therapeutic beam of the controlled spot size into the eyeball.

11. The method according to claim 10, wherein the step of irradiating the controlled therapeutic beam into the eyeball comprises using a second lens part that converges the therapeutic beam of which focal length has been varied by the first lens part to transfer the therapeutic beam thereto.

12. The method according to claim 10, wherein the step of controlling the spot size of the therapeutic beam to be formed comprises generating a variable signal based on an amount of current or voltage in the control unit; and transmitting the variable signal to the first lens unit so that the spot size of the therapeutic beam is varied.

13. The method according to claim 10, further comprising setting an irradiation position of the therapeutic beam transferred to a treatment region of the eyeball prior to the step of controlling the predetermined spot size to be formed in the eyeball.
